(19) United States
(12) Patent Application Publication
(10) Pub. No.: US 2004/0110756 A1
(43) Pub. Date: Jun. 10, 2004

(54) New anthracene derivatives and their use as pharmaceutical preparations

(76) Inventors: Peter Emig, Bruchkobel (DE); Eckhard Gunther, Maintal (DE); Beate Aue, Grossostheim-Ringheim (DE); Emmanuel Polymeropoulos, Frankfurt/Ma in (DE); Silke Baasner, Schoneck (DE); Peter Schmidt, Schoneck (DE)

Correspondence Address: GOODWIN PROCTER L.L.P, 103 EISENHOWER PARKWAY, ROSELAND, NJ, 07068, US

(21) Appl. No.: 10/621,590

(22) Filed: Jul. 17, 2003

Related U.S. Application Data
(60) Provisional application No. 60/396,683, filed on Jul. 17, 2002.

(51) Int. Cl. A61K 31/5377; A61K 31/495; C07D 413/02

(52) U.S. Cl. 514/238.5; 514/255.01; 544/120; 544/380

(57) ABSTRACT

The invention relates to new anthracene derivatives of the general Formula 1 and to their synthesis and use as pharmaceutical preparations, especially for the treatment of a tumor.

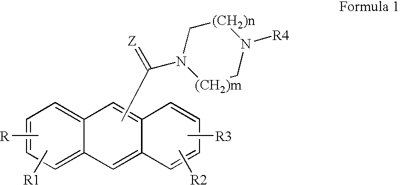

### CROSS-REFERENCE TO RELATED APPLICATIONS

This application claims priority from U.S. Provisional Application No. 60/396,683 filed Jul. 17, 2002, which is hereby incorporated by reference in its entirety.

### BACKGROUND OF THE INVENTION

1. Field of Invention

The present invention relates to anthracene derivatives and uses as pharmaceutical preparations.

2. Related Background Art

For the next few years, a dramatic increase in tumor diseases and tumor-induced deaths is expected worldwide. In 2001, about 10 million people worldwide contracted cancer and more than 6 million people died from this illness. The development of tumors is a fundamental illness of higher organisms in the plant and animal kingdoms and in man. The generally recognized multi-step model of the development of cancer starts out from the fact that, due to the accumulation of several mutations in a single cell, the latter is so changed in its proliferation and differentiation behavior, that finally, by way of benign intermediate stages, a malignant condition with metastasizing is reached. Behind the concept of cancer or tumor, a clinical picture is concealed with more than 200 different individual diseases. Tumor diseases can be benign or malignant. The most important tumors are those of the lung, the breast, the stomach, the cervix, the prostate, the head and neck, the large intestines and the rectum, the liver and the blood system. With regard to the course, prognosis and therapy behavior, there are large differences. More than 90% of the identified cases relate to solid tumors, which can be treated only with difficulty, if at all, especially in advanced stages or after metastacizing. The three pillars of combating cancer continue to be surgical removal, radiation and chemotherapy. In spite of great progress, it has not yet been possible to develop pharmaceutical drugs, which, in the case of widespread, solid tumors, bring about a clear prolongation in survival time or even complete healing. It therefore makes sense to discover new pharmaceutical preparations for combating cancer diseases.

Aryl and heteroaryl-substituted piperazinylcarbonyl derivatives have various uses as pharmacologically active compounds and as synthesis building blocks in pharmaceutical chemistry. For example, in the application WO 200059930, 2-alkyl-1-(1H-imidazole-1-yl)-4-(1-naphthalenecarbonyl)-piperazines are described, which are used for the treatment of prostate cancer. In the application WO 9714685, derivatives of the 1-(pyrimidinyl)-4-(naphthyl-carbonyl)-piperazine series are mentioned, which are used as lipid peroxidation inhibitors. D. L. Romero et al. (Upjohn Laboratories) gave a report in J. Med. Chem. 37, 999 (1994) about 1-pyridinyl-4-(naphthylcarbonyl)-piperazines, which are characterized as HIV-1-reverse transcriptase inhibitors. In the U.S. Pat. No. 5,110,927, 1-(quinazolinyl)-4-(naphthylcarbonyl)-piperazines are profiled as prazosine analogs with α-1-adrenergic-blocking and anti-hypertensive effects. In the J. Med. Chem., S. Sharma et al. describe the synthesis and the anthelminitic activity of 1-(3,4-diaminophenyl)-4-((1-hydroxy-2-naphthyl)carbonyl)-piperazines, as well as of 1-(benzimidazolyl)-4-(naphthyl-carbonyl)-piperazines

### BRIEF SUMMARY OF INVENTION

The present invention relates to new aryl-substituted and heteroaryl-substituted piperazinylcarbonyl aromatic compounds, their synthesis and use as pharmaceutical preparations, especially for treating benign and malignant tumors in man and mammals.

### DESCRIPTION OF THE INVENTION

It has now surprisingly been found that new compounds of the aryl-substituted and heteroaryl-substituted aromatic piperazinylcarbonyl compounds are suitable for the synthesis of pharmaceutical preparations, which, in particular, are suitable for the treatment of benign and malignant tumors. In accordance with this point of view, new compounds are described in the present application from the series of aryl-substituted and heteroaryl-substituted piperazinylcarbonyl anthracenes of the general Formula 1

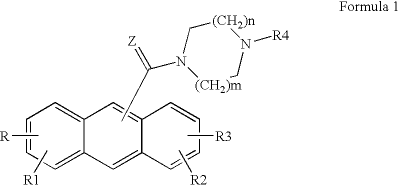

in which

R, R₁, R₂, R₃ may be linked to the C₁ to C₁₀ aromatic carbon atoms of anthracene, alternatively are the same or different and, independently of one another, represent hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably allyl, (C₂-C₆)-alkinyl, preferably ethinyl or propargyl, aryl, the aryl group being unsubstituted or a linear or branched (C₁-C₈)-alkyl, substituted one or more times, similarly or differently, with halogen, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, cyano, isocyano, linear or branched (C₁-C₈)-alkoxycarbonyl, linear or branched (C₁-C₄)-alkyl substituted with one or more halogen atoms, preferably trifluoromethyl, (C₂-C₆)-alkenyl, preferably allyl, (C₂-C₆)-alkinyl, preferably ethinyl or propargyl,

Z is oxygen or sulfur, the

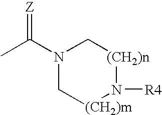

group, which is a substituent on the anthracene compound, may be linked to any one of the C₁-C₁₀ carbon atoms of the ring structure;

n, m independently of one another, each represent a whole number from 1 to 4;

R₄ is a linear or branched (C₁-C₁₂)-alkyl group, which is saturated or unsaturated with one to three double bonds and/or triple bonds and is unsubstituted or alternatively substituted at the same or at different carbon atoms with one, two or more aryl, heteroaryl, halogen, cyano, (C₁-C₆)-alkoxycarbonylamino, (C₁-C₆)-alkoxy, amino, mono-(C₁-C₄)-alkylamino or di-(C₁-C₄)-alkylamino, a (C₆-C₁₄)-aryl group, (C₆-C₁₄)-aryl-(C₁-C₄)-alkyl group or a (C₂-C₁₀)-heteroaryl or (C₂-C₁₀)-heteroaryl-(C₁-C₄)-alkyl group, containing one or more hetero atoms selected from the group comprising nitrogen, oxygen and sulfur, the (C₁-C₄)-alkyl group being unsubstituted or substituted one or more times, identically or differently with (C₁-C₆)-alkyl or halogen and the (C₆-C₁₄)-aryl or (C₂-C₁₀)-heteroaryl group represent unsubstituted or substituted, one or more times with the same or different linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl.

Provided that the inventive compounds of the general Structure 1 have at least one center of asymmetry, they can be present in the form of their racemates, in the form of the pure enantiomers and/or diastereoisomers or in the form of mixtures of these enantiomers and/or diastereoisomers.

The compounds of this invention may also be present in the form of the tautomers.

For example, the inventive compounds of the general Formula 1, which have one or more centers of chirality and which occur as racemates, can be separated by known methods into their optical isomers, that is, enantiomers or diastereoisomers. The separation can be brought about by column separation on chiral phases or by recrystallization from an optically active solvent or using an optically active acid or base or by forming a derivative with an optically active reagent, such as, for example, an optically active alcohol, and subsequently splitting off the group.

The inventive compounds of the general Formula 1, provided that they have a sufficiently basic group, such as a secondary or tertiary amine, can be converted with inorganic or organic acids into salts. Preferably, the pharmaceutically acceptable salts of the inventive compounds of the general Structure 1 are formed with hydrochloric acid, hydrobromic acid, sulfuric acid, phosphoric acid, methanesulfonic acid, p-toluenesulfonic acid, carbonic acid, formic acid, acetic acid, trifluoroacetic acid, oxalic acid, malonic acid, maleic acid, succinic acid, tartaric acid, malic acid, embonic acid, mandelic acid, fumaric acid, lactic acid, citric acid, glutamic acid or aspartic acid. The salts formed are, for example, hydrochlorides, hydrobromides, sulfates, phosphates, methanesulfonates, sulfoacetates, tosylates, carbonates, hydrogen carbonates, formates, acetates, trifluoroacetates, oxalates, malonates, maleates, succinates, tartrates, malates, embonates, mandelates, fumarates, lactates, citrates and glutaminates. The stoichiometry of the salts formed from the inventive compounds can be a whole number, or a fractional number which is not a whole number.

The inventive compounds of the general Formula 1, in the event that they contain a sufficiently acidic group, such as the carboxy group, can be converted with an inorganic or organic base into a physiologically tolerated salt. As inorganic bases, sodium hydroxide, potassium hydroxide and calcium hydroxide and, as organic bases, ethanolamine, diethanolamine, triethanolamine, cyclohexylamine, dibenzylethylenediamine and lysine, for example, come into consideration. The stoichiometry of the salts formed from the inventive compounds can be a whole number, or a fractional number which is not a whole number.

Also preferred are solvates and, in particular, hydrates of the inventive compounds, which can be obtained, for example, by crystallization from a solvent or from aqueous solution. Moreover, one, two, three or any number of solvate or water molecules can combine with the inventive compounds to form solvates and hydrates.

It is well known that chemical substances form solids, which can be present in different arrangements, which are referred to as polymorphic forms or modifications. The physical properties of the different modifications of a polymorphic substance can differ greatly. The inventive compounds of the general Formula 1 can be present in different polymorphic forms and certain modifications may be metastable.

Pursuant to a preferred embodiment, anthracene derivatives of the general Formula 1 are prepared, in which R, R₁, R₂, R₃, Z, n and m have the meanings given above and

R₄ represents a phenyl or a naphthyl group, which is unsubstituted or substituted once or repeatedly with the same or different linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 2-, 4-, 5- or 6-pyrimidinyl group or a 2-, 4-, 5- or 6-pyrimidinyl-(C₁-C₄)-alkyl group, the (C₁-C₄)-alkyl group being unsubstituted or substituted, once or repeatedly, identically or differently, with (C₁-C₆)-alkyl or halogen, and the 2-, 4-, 5- or 6-pyrimidinyl group being unsubstituted or substituted one to three times with the same or different, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl; a 3-, 4-, 5- or 6-pyridazinyl group or a 3-, 4-, 5- or 6-pyridazinyl-(C₁-C₄)-alkyl group, the (C₁-C₄)-alkyl group being unsubstituted or substituted one or more times, identically or differently, with linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 2-, 3-, 5- or 6-pyrazinyl group or a 2-, 3-, 5- or 6-pyrazinyl-(C₁-C₄)-alkyl group, the (C₁-C₄)-alkyl group being unsubstituted or substituted one or more times, identically or differently, with (C₁-C₆)-alkyl, halogen and the 2-, 3-, 5-, or 6-pyrazinyl group being unsubstituted or substituted one to three times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 3-, 4-, 5-, 6-, 7- or 8-cinnolinyl group or a 3-, 4-, 5-, 6-, 7- or 8-cinnolinyl-(C₁-C₄)-alkyl group, the (C₁-C₄)-alkyl group being unsubstituted or substituted one or more times, identically or differently, with (C₁-C₆)-alkyl, halogen and the 3-, 4-, 5-, 6-, 7- or 8-cinnolinyl group being unsubstituted or substituted one to five times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 2-, 4-, 5-, 6-, 7- or 8-quinazolinyl group or a 2-, 4-, 5-, 6-, 7- or 8-quinazolinyl-(C₁-C₄)-alkyl group, the (C₁-C₄)-alkyl group being substituted one or more times, identically or differently, with hydrogen, (C₁-C₆)-alkyl, halogen, and the 2-, 4-, 5-, 6-, 7- or 8-quinazolinyl group being substituted one to five times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 2-, 3-, 5-, 6-, 7- or 8-quinoxalinyl group or a 2-, 3-, 5-, 6-, 7- or 8-quinoxalinyl-(C₁-C₄)-alkyl group, the (C₁-C₄)-alkyl group being unsubstituted or substituted one or more times, identically or differently, with (C₁-C₆)-alkyl or halogen and the 2-, 3-, 5-, 6-, 7- or 8-quinoxalinyl group being unsubstituted one to five times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 1-, 4-, 5-, 6-, 7-, or 8-phthalazinyl group or a 1-, 4-, 5-, 6-, 7-, or 8-phthalazinyl-(C₁-C₄)-alkyl group, the (C₁-C₄)-alkyl group being unsubstituted or substituted one or more times, identically or differently, with (C₁-C₆)-alkyl or halogen and the 1-, 4-, 5-, 6-, 7- or 8-phthalazinyl group being substituted one to five times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, being substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 2-, 3-, 4-, 5-, 6-, 7- or 8-quinolyl group or a 2-, 3-, 4-, 5-, 6-, 7- or 8-quinolyl-(C₁-C₄)-alkyl group, the (C₁-C₄)-alkyl group being unsubstituted or substituted one or more times, identically or differently, with (C₁-C₆)-alkyl or halogen, and the 2-, 3-, 4-, 5-, 6-, 7- or 8-quinolyl group being substituted one to six times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, being substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 1-, 3-, 4-, 5-, 6-, 7- or 8-isoquinolyl or a 1-, 3-, 4-, 5-, 6-, 7- or 8-isoquinolyl-(C₁-C₄)-alkyl group, the (C₁-C₄)-alkyl group being unsubstituted or substituted one or more times, identically or differently, with (C₁-C₆)-alkyl or halogen, and the 1-, 3-, 4-, 5-, 6-, 7- or 8-isoquinolyl group being substituted one to six times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, being substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 2-, 6-, 8- or 9-[9H]-purinyl group or 2-, 6-, 8- or 9-[9]H-purinyl-(C₁-C₄)-alkyl group, the (C₁-C₄)-alkyl group being unsubstituted or substituted one or more times, identically or differently, with (C₁-C₆)-alkyl or halogen, and the 2-, 6-, 8- or 9-[9H]-purinyl group being substituted one to three times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 2-, 6-, 7- or 8-[7H]-purinyl group or 2-, 6-, 7- or 8-[7H]-purinyl-(C₁-C₄)-alkyl group, the (C₁-C₄)-alkyl group being unsubstituted or substituted one or more times, identically or differently with (C₁-C₆)-alkyl or halogen, and the 2-, 6-, 7- or 8-[7H]-purinyl group being substituted one to three times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 1-, 2-, 3-, 4-, 5-, 6-, 7-, 8- or 9-acridinyl or a 1-, 2-, 3-, 4-, 5-, 6-, 7-, 8- or 9-acridinyl-(C₁-C₄)-alkyl group, the (C₁-C₆)-alkyl group being unsubstituted or substituted one or more times, identically or differently, with (C₁-C₆)-alkyl or halogen and the 1-, 2-, 3-, 4-, 5-, 6-, 7-, 8- or 9-acridinyl group being substituted one to eight times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 1-, 2-, 3-, 4-, 5-, 6-, 7-, 8 or 9-phenanthridinyl or a 1-, 2-, 3-, 4-, 5-, 6-, 7-, 8- or 9-phenanthridinyl-(C₁-C₆)-alkyl group, the (C₁-C₆)-alkyl group being unsubstituted or substituted one or more times, identically or differently with hydrogen, (C₁-C₆)-alkyl, or halogen, and the 1-, 2-, 3-, 4-, 5-, 6-, 7-, 8- or 9-phenanthridinyl group being unsubstituted or substituted one to eight times, identically or differently with linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 2-, 3-, 4-, 5- or 6-pyridyl group, the 2-, 3-, 4-, 5- or 6-pyridinyl group being substituted one to four times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 2-, 3-, 4-, 5- and 6-pyridyl-(C₁-C₆)-alkyl group, the (C₁-C₆)-alkyl group being unsubstituted or substituted one or more times, identically or differently, with (C₁-C₆)-alkyl or halogen, and the 2-, 3-, 4-, 5- or 6-pyridinyl group being substituted one to four times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 2-, 3-, 4- or 5-thienyl group or a 2-, 3-, 4- or 5-thienyl-(C₁-C₆)-alkyl group, the (C₁-C₆)-alkyl group being unsubstituted or substituted one or more times, identically or differently, with (C₁-C₆)-alkyl or halogen and the 2-, 3-, 4- or 5-thienyl group being substituted one to three times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 2-, 4- or 5-thiazolyl group or a 2-, 4- or 5-thiazolyl-(C₁-C₆)-alkyl group, the (C₁-C₆)-alkyl group being unsubstituted or substituted one or more times, identically or differently, with (C₁-C₆)-alkyl or halogen and the 2-, 4- or 5-thiazolyl group being substituted once or twice, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, with or more substituted halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 3-, 4- or 5-isothiazolyl group or a 3-, 4- or 5-isothiazolyl-(C₁-C₆)-alkyl group, the (C₁-C₆)-alkyl group being unsubstituted or substituted one or more times, identically or differently with (C₁-C₆)-alkyl or halogen, and the 3-, 4- or 5-isothiazolyl group being substituted once or twice, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 2-, 4-, 5-, 6- or 7-benzthiazolyl group or a 2-, 4-, 5-, 6- or 7-benzthiazolyl-(C₁-C₆)-alkyl group, the (C₁-C₆)-alkyl group being unsubstituted or substituted one or more times, identically or differently, with (C₁-C₆)-alkyl or halogen, and the 2-, 4-, 5-, 6- or 7-benzthiazolyl group being substituted one to four times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 1-, 2-, 4- or 5-imidazolyl group or a 1-, 2-, 4- or 5-imidazolyl-(C₁-C₆)-alkyl group, the (C₁-C₆)-alkyl group being unsubstituted or substituted one or more times, identically or differently, with (C₁-C₆)-alkyl or halogen and the 1-, 2-, 4- or 5-imidazolyl group being substituted one to three times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 1-, 3-, 4- or 5-pyrazolyl group or a 1-, 3-, 4- or 5-pyrazolyl-(C₁-C₆)-alkyl group, the (C₁-C₆)-alkyl group being unsubstituted or substituted one or more times, identically or differently, with (C₁-C₆)-alkyl or halogen, and the 1-, 3-, 4- or 5-pyrazolyl group being substituted one to three times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 1-, 2-, 3-, 4- or 5-pyrrolyl group or a 1-, 2-, 3-, 4- or 5-pyrrolyl-(C₁-C₆)-alkyl group, the (C₁-C₆)-alkyl group being unsubstituted or one or more times, identically or differently, with (C₁-C₆)-alkyl or halogen, and the 1-, 2-, 3-, 4- or 5-pyrrolyl group being substituted one to four times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, with or more substituted halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 1-, 3- or 5-[1,2,4]-triazolyl group or 1-, 3- or 5-[1,2,4]-triazolyl-(C₁-C₆)-alkyl group, the (C₁-C₆)-alkyl group being substituted one or more times, identically or differently, with hydrogen, (C₁-C₆)-alkyl or halogen and the 1-, 3- or 5-[1,2,4]-triazolyl group, unsubstituted or substituted once or twice, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 1,4- or 5-[1.2.3]-triazolyl group or a 1-, 4- or 5-[1.2.3]-triazolyl-(C₁-C₆)-alkyl group, the (C₁-C₆)-alkyl group being unsubstituted or substituted one or more times, identically or differently, with (C₁-C₆)-alkyl or halogen and the 1-, 4- or 5-[1.2.3]-triazolyl group being substituted once or twice, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 1- or 5-[1H]-tetrazolyl group or a 1- or 5-[1H]-tetrazolyl-(C₁-C₆)-alkyl group, the (C₁-C₆)-alkyl group being unsubstituted or substituted one or more times, identically or differently, with (C₁-C₆)-alkyl or halogen, and the 1- or 5-[1H]-tetrazolyl group being substituted with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 2- or 5-[2H]-tetrazolyl group or a 2- or 5-[2H]-tetrazolyl-(C₁-C₆)-alkyl group, the (C₁-C₆)-alkyl group being unsubstituted or substituted one or more times, identically or differently, with (C₁-C₆)-alkyl or halogen, and the 2- or 5-[2H]-tetrazolyl group being substituted with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 2-, 4- or 6-[1.3.5]-triazinyl group or a 2-, 4- or 6-[1.3.5]-triazinyl-(C₁-C₆)-alkyl group, the (C₁-C₆)-alkyl group being unsubstituted or substituted one or more times, identically or differently, with hydrogen, (C₁-C₆)-alkyl or halogen and the 2-, 4- or 6-[1.3.5]-triazinyl group being substituted once or twice, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, with or more substituted halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 2-, 4- or 5-oxazolyl group or a 2-, 4- or 5-oxazolyl-(C₁-C₆)-alkyl group, the (C₁-C₆)-alkyl group being unsubstituted or substituted one or more times, identically or differently, with (C₁-C₆)-alkyl or halogen, and the 2-, 4- or 5-oxazolyl group being substituted once or twice, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl; a 3-, 4- or 5-isoxazolyl group or a 3-, 4- or 5-isoxazolyl-(C₁-C₆) group, the (C₁-C₆)-alkyl group being unsubstituted or substituted one or more times, identically or differently, with (C₁-C₆)-alkyl or halogen, and the 3-, 4- or 5-isoxazolyl group being substituted once or twice, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;

a 1-, 2-, 3-, 4-, 5-, 6- or 7-indolyl group or a 1-, 2-, 3-, 4-, 5-, 6- or 7-indolyl-(C₁-C₆)-alkyl group, the (C₁-C₆)-alkyl group being unsubstituted or substituted one or more times, identically or differently, with (C₁-C₆)-alkyl or halogen, and the 1-, 2-, 3-, 4-, 5-, 6- or 7-indolyl group being substituted one to six times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, I preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl

Pursuant to a further embodiment, anthracene derivatives of the general Formula 1 are prepared, wherein R, R₁, R₂, R₃, Z, n and m have the meanings given above and R₄ represents phenyl, which is unsubstituted or substituted with one to five identical or different (C₁-C₆)-alkoxy groups. Neighboring oxygen atoms can also be linked by (C₁-C₂) alkylene groups.

Pursuant to a further embodiment, anthracene derivatives of the general Formula 1 are prepared, wherein R, R₁, R₂, R₃, Z, n and m have the meanings given above and R₄ represents 3,5-dimethoxyphenyl.

Pursuant to a further embodiment, anthracene derivatives of the general Formula 1 are prepared, wherein R₄ and Z have the meanings given above, R, R₁, R₂, R₃, represent hydrogen atoms, m is 1 and n represents the whole numbers 1 and 2.

The compounds of the general Formula 1, which are given below, are the ones preferred most:

Anthracene-9-yl-[4-(4-nitro-phenyl)-piperazine-1-yl]-methanone (1)

Anthracene-9-yl-[4-(3,5-dimethoxy-phenyl)-piperazine-1-yl]-methanone (2)

Anthracene-9-yl-[4-phenyl)-piperazine-1-yl]-methanone (3)

Anthracene-9-yl-(4-naphthalene-1-yl-piperazine-1-yl)-methanone (4)

Anthracene-9-yl-(4-biphenyl-2-yl-piperazine-1-yl)-methanone (5)

Anthracene-9-yl-[4-(3-hydroxy-phenyl)-piperazine-1-yl]-methanone (6)

Anthracene-9-yl-[4-(4-trifluoromethyl-pyridine-2-yl)-piperazine-1-yl]-methanone (7)

Anthracene-9-yl-[4-(6-methyl-pyridine-2-yl)-piperazine-1-yl]-methanone (8)

Anthracene-9-yl-(2,3,5,6-tetrahydro-[1,2′]-bipyrazinyl-4-yl)-methanone (9)

2-[4-(Anthracene-9-carbonyl)-piperazine-1-yl]-nicotinnitrile (10)

Anthracene-9-yl-[4-(5-trifluoromethyl-pyridine-2-yl)-piperazine-1-yl]-methanone (11)

Anthracene-9-yl-(4-pyridine-2-yl-piperazine-1-yl)-methanone (12)

Pursuant to a further embodiment, anthracene derivatives of the general Formula 1 are prepared, wherein R, R₁, R₂ and R₃ represent hydrogen atoms, Z has the meanings given above, m and n represent the whole number 1 and R₄ represents a 3,5-dimethoxyphenyl group.

Pursuant to a further aspect of the invention, a method is claimed for the synthesis of anthracene derivatives of one of the claims 1 to 6, wherein an anthracenecarboxylic acid of the general Formula 2, in which R, R₁, R₂, R₃ have the meanings given above, Z represents an oxygen or sulfur atom and Y represents a leaving group, such as halogen, hydroxy, (C₁-C₆)-alkoxy, preferably methoxy and ethoxy, —O-tosyl, —O-mesyl, tetrazolyl or imidazolyl,

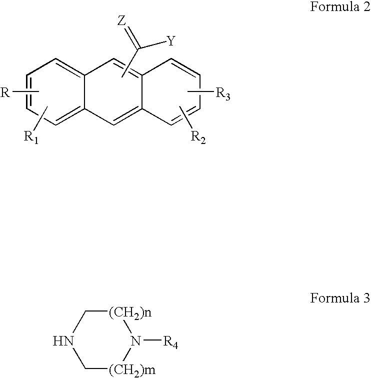

is reacted with an amine of the general Formula 3, in which R₄, m and n have the meanings given above, optionally using a condensing agent and/or a catalyst, as well as diluents and adjuvants, with the formation of the desired anthracene derivatives.

Synthesis of the Inventive Compounds

The compounds of the general formula 1 may be obtained, for example, according to the following outline 1:

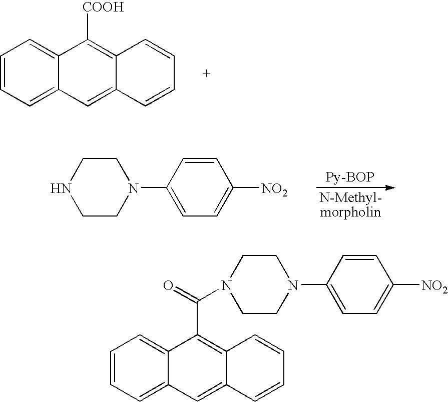

The starting materials 2 and 3 can either be obtained commercially or synthesized by known procedures. The educts 2 and 3 represent valuable intermediates for the synthesis of the inventive anthracene derivatives of Formula 1.

The solvents and adjuvants, which may be used, and the reaction parameters, such as the reaction temperature and duration, are known to someone skilled in the art.

The inventive anthracene derivatives of the general Formula 1, as active ingredients in pharmaceutical preparations, are suitable especially as anti-tumor agents for the treatment of man and mammals. The mammals may be domestic animals, such as horses, cows, dogs, cats, rabbits, sheep, poultry and the like.

The medicinal effect of the inventive anthracene derivatives may be based, for example, on the interaction with the tubulin system due to the inhibiting of the tubulin polymerization. In addition, further known and unknown mechanisms of action for combating tumor cells are conceivable.

Pursuant to a further aspect of the invention, a method for combating tumors in man and mammals is made available, wherein at least one anthracene derivative of the general Formula 1 is administered to the person or a mammal in an amount, which is effective for the treatment of the tumor. The therapeutically effective dose of the respective, inventive, anthracene derivatives, which is to be administered for the treatment, depends, for example, on the nature and stage of the tumor disease, the age and sex of the patient, the nature of the administration and the duration of the treatment, and is known to those of skill in the art. The inventive pharmaceutical preparation can be administered as a liquid, semi-solid or solid drug form as aerosols, powders and dusting powders, tablets, coated pillows, emulsions, foams, solutions, suspensions, gels, ointments, pastes, pills, lozenges, capsules or suppositories.

Aside from at least one inventive component, the drug forms optionally contain, depending on the pharmaceutical form used, adjuvants, such as solvents, dissolving accelerators, solubilizers, emulsifiers, wetting agents, defoamers, gel-forming agents, thickeners, film-forming agents, binders, buffers, salt-forming agents, drying agents, flow regulators, fillers, preservatives, antioxidants, dyes, mold-release agents, lubricants, disintegrants and taste- and odor-correcting substances. The type of adjuvant to be selected and the amount used depends on the pharmaceutical form chosen and is guided by formulations known to those skilled in the art.

The inventive pharmaceutical preparation can be administered in a suitable form on the skin, epicutaneously as a solution, suspension, emulsion, foam, ointment, paste or plaster, by way of the mucous membrane of the mouth and the tongue, buccally, lingually or sublingually as a tablet, lozenge, coated pill, tincture or gargle, by way of the gastric and the intestinal mucosa, enterally as a tablet, coated tablet, capsule, solution, suspension, or emulsion, by way of the mucous membrane of the rectum as a suppository, rectal capsule or ointment by way of mucous membrane of the nose, nasally as droplets, ointment or spray, by way of the epithelium of the bronchia and alveoli, pulmonally or by inhalation as an aerosol or inhalant, by way of the conjunctiva, conjunctivally as eye drops, eye ointment, eye tablets, lamellae or eye lotion, by way of the mucous membranes of the genital organs, intravaginally as vaginal suppositories, ointments and douches, intrauterinally as a pessary for supporting the uterus, by way of urinary draining tracts, intraurethrally as a wash, ointment or small medicinal rod, in an artery, intraarterially as an injection in a vein intravenously as an injection or infusion, into the hand, intracutaneously as an injection or implant, under the skin, subcutaneously or as an implant into the muscle, intramuscularly as an injection or implant, into the abdominal cavity, intraperitoneally, as an injection or infusion.

The period of action of the inventive compounds of the general Structure 1 can be prolonged by suitable measures with respect to practical therapeutic requirements. This objective can be achieved by chemical and pharmaceutical means. Examples of achieving a prolongation of the action are the use of implant and liposomes, the formation of salts and complexes of low solubility or the use of crystalline suspensions.

Particularly preferred, in this connection, are drugs, which contain at least one compound from the following group of aryl derivatives:

Anthracene-9-yl-[4-(4-nitro-phenyl)-piperazine-1-yl]-methanone (1)

Anthracene-9-yl-[4-(3,5-dimethoxy-phenyl)-piperazine-1-yl]-methanone (2)

Anthracene-9-yl-[4-phenyl)-piperazine-1-yl]-methanone (3)

Anthracene-9-yl-(4-naphthalene-1-yl-piperazine-1-yl)-methanone (4)

Anthracene-9-yl-(4-biphenyl-2-yl-piperazine-1-yl)-methanone (5)

Anthracene-9-yl-[4-(3-hydroxy-phenyl)-piperazine-1-yl]-methanone (6)

Anthracene-9-yl-[4-(4-trifluoromethyl-pyridine-2-yl)-piperazine-1-yl]-methanone (7)

Anthracene-9-yl-[4-(6-methyl-pyridine-2-yl)-piperazine-1-yl]-methanone (8)

Anthracene-9-yl-(2,3,5,6-tetrahydro-[1,2′]-bipyrazinyl-4-yl)-methanone (9)

2-[4-(Anthracene-9-carbonyl)-piperazine-1-yl]-nicotinnitrile (10)

Anthracene-9-yl-[4-(5-trifluoromethyl-pyridine-2-yl)-piperazine-1-yl]-methanone (11)

Anthracene-9-yl-(4-pyridine-2-yl-piperazine-1-yl)-methanone (12)

and can be present as free base and as salt of physiologically tolerated acids.

The invention will be described in greater detail by means of the following examples which are not intended to limit the invention.

### EXAMPLE I

1-(4-Nitrophenyl)-4-(anthracene-9-yl-carbonyl) piperazine

A solution of 1 g (4.45 mmoles) of anthracene-9-carboxylic acid in 30 mL of dimethylformamide was treated consecutively with 0.73 g (7.2 mmoles) of N-methylmorpholine, 0.932 g (4.49 mmoles) of 1-(4-nitrophenyl)-piperazine and 2.57 g (4.94 mmoles) of Py-BOP (1-benzotriazolyltripyrrolidinophosphonium hexafluorophosphate). The mixture was stirred for 4 hours at room temperature and allowed to stand overnight at room temperature. The dimethylformamide was then distilled off under vacuum and the residue purified on a silica column (Kieselgel 60, Fa. Merck AG, Darmstadt) using a 95:5 (v/v) mixture of dichloromethane and methanol as eluent.

Yield: 1.46 g (79.7% of the theoretical yield)

M.P.: 278°

The following compounds of the general Formula 1 were synthesized by a procedure similar to that shown in outline 1 above:

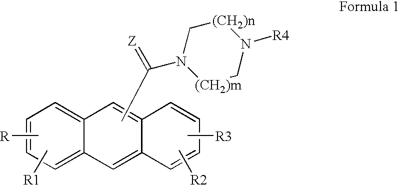

TABLE 1
Anthracene Derivative Compounds with Anti-tumor Effect

| | | m/e |
|---|---|---|
| Example | Name | (M + H) |
| 1 | Anthracene-9-yl-[4-(4-nitro-phenyl)-piperazine- | |
| | 1-yl]-methanone (1) | |
| 2 | Anthracene-9-yl-[4-(3,5-dimethoxy-phenyl)- | 427 |
| | piperazine-1-yl]-methanone (2) | |
| 3 | Anthracene-9-yl-[4-phenyl)-piperazine-1- | 367 |
| | yl]-methanone (3) | |
| 4 | Anthracene-9-yl-(4-naphthalene-1-yl-piperazine- | 417 |
| | 1-yl)-methanone (4) | |
| 5 | Anthracene-9-yl-(4-biphenyl-2-yl-piperazine- | 443 |
| | 1-yl)-methanone (5) | |
| 6 | Anthracene-9-yl-[4-(3-hydroxy-phenyl)- | 383 |
| | piperazine-1-yl]-methanone (6) | |
| 7 | Anthracene-9-yl-[4-(4-trifluoromethyl-pyridine- | 436 |
| | 2-yl)-piperazine-1-yl]-methanone (7) | |
| 8 | Anthracene-9-yl-[4-(6-methyl-pyridine-2-yl)- | 382 |
| | piperazine-1-yl]-methanone (8) | |
| 9 | Anthracene-9-yl-(2,3,5,6-tetrahydro-[1,2′]- | 369 |
| | bipyrazinyl-4-yl)-methanone (9) | |
| 10 | 2-[4-(Anthracene-9-carbonyl)-piperazine-1- | 393 |
| | yl]-nicotinnitrile (10) | |
| 11 | Anthracene-9-yl-[4-(5-trifluoromethyl-pyridine- | 436 |
| | 2-yl)-piperazine-1-yl]-methanone (11) | |
| 12 | Anthracene-9-yl-(4-pyridine-2-yl-piperazine-1- | 368 |
| | yl)-methanone (12) | |

The most preferred compounds of the present invention are substances of the general Formula 1 in the form of their bases or pharmaceutically acceptable salts, selected from the following group:

Anthracene-9-yl-[4-(4-nitro-phenyl)-piperazine-1-yl]-methanone (1)

Anthracene-9-yl-[4-(3,5-dimethoxy-phenyl)-piperazine-1-yl]-methanone (2)

Anthracene-9-yl-[4-phenyl)-piperazine-1-yl]-methanone (3)

Anthracene-9-yl-(4-naphthalene-1-yl-piperazine-1-yl)-methanone (4)

Anthracene-9-yl-(4-biphenyl-2-yl-piperazine-1-yl)-methanone (5)

Anthracene-9-yl-[4-(3-hydroxy-phenyl)-piperazine-1-yl]-methanone (6)

Anthracene-9-yl-[4-(4-trifluoromethyl-pyridine-2-yl)-piperazine-1-yl]-metharione (7)

Anthracene-9-yl-[4-(6-methyl-pyridine-2-yl)-piperazine-1-yl]-methanone (8)

Anthracene-9-yl-(2,3,5,6-tetrahydro-[1,2′]-bipyrazinyl-4-yl)-methanone (9)

2-[4-(Anthracene-9-carbonyl)-piperazine-1-yl]-nicotinnitrile (10)

Anthracene-9-yl-[4-(5-trifluoromethyl-pyridine-2-yl)-piperazine-1-yl]-methanone (11)

Anthracene-9-yl-(4-pyridine-2-yl-piperazine-1-yl)-methanone (12)

Biological Effects of the Inventive Compounds

The in vitro testing of selected tumor models revealed the following pharmacological activities.

Antiproliferative Effect on Different Tumor Cell Lines

The substance of Example 2 was investigated in a proliferation test on established tumor cell lines for its anti-proliferative activity. The test used determines the cellular dehydrogenase activity and enables the cell vitality and, indirectly, the cell count to be determined. The cell lines used are the human cervix carcinoma cell line KB/HeLa (ATCC CCL17), the ovarian adenocarcinoma cell line SKOV-3 (ATCC HTB77), the human glioblastoma cell line SF-268 (NCI 503138) and the lung carcinoma cell line NCI-H460 (NCI 503473). Moreover, for investigating the effect of the substance specifically on the cell cycle, an RKOp27 cell system was used (M. Schmidt et al. Oncogene 19(20):2423-9, 2000). The RKO is a human colon carcinoma line, in which the cell cycle inhibitor p27^{kip1} was expressed by means of the Ecdyson Expression System and can be brought to a cell cycle arrest in G2. A substance of nonspecific activity inhibits the proliferation independently of whether the RKO cell is or is not arrested in G1 or G2. On the other hand, cell cycle specific substances, such as tubulin inhibitors, are cytotoxic only when the cells are not arrested and passed through the cell cycle. In Table 2, the cytotoxic or growth-inhibiting activity of the compound described is shown with and without expression of p27^{kip1}. In the induced state of p27^{kip1}, the compound tested did not show any cytotoxic activity. The results show a very potent inhibition of the proliferation of selected tumor cell line by the substances named as Example 2.

TABLE 2
Proliferation prohibition of substance 2 in XTT in the cytotoxicity
test carried out with human tumor cell lines.

| Example | XTT Proliferation Assay, EC50 in μg/mL | | | | | |
|---|---|---|---|---|---|---|
| | KB/ | SKOV/ | SF-268 | NCI-H460 | RKOP27 | RKOP27 |
| | Hela | 3 | | | | ind. |
| 2 | 0.048 | 0.047 | 0.043 | 0.054 | 0.058 | >3.16 |

For further characterization, substance 2 was investigated for its anti-proliferative effect using a panel of 12 human tumor cell lines. The cell lines used were the human glioma cell lines A172 (ATCC CRL-1620), U118 (ATCC HTB-15) and U373 (ATCC HTB-17), the rat glioma cell line C6 (ATCC CCL-107), the human lung carcinoma cell line A549, the human epidermoid carcinoma A431 (ATCC CRL-2592), the human prostate cancer cell lines DU145 (ATCC HTB-81) and PC3 (ATCC CRL-1435), the human colon adenocarcinoma cell line HT 29 (ATCC HTB-38), the human pancreas adenocarcinoma cell line ASPC1 (ATCC CRL-1682) and the human breast cancer cell lines T47D (ECACC 88062428) and MCAMB435 (NCI 914182). These are well-characterized, established cell lines, which are maintained by ATCC, ECACC and NCI and from which cultures were taken. The results, summarized in Table 3, show a very potent inhibiting effect of substance 2 on all cell lines tested.

TABLE 3
Inhibiting effect of substance 2 in the XTT proliferation tests on
human tumor cell lines. The average values of two independent
experiments are given.

| Example | XTT Proliferation Assay, EC50 in μg/mL | | | | | |
|---|---|---|---|---|---|---|
| | ASPC1 | A172 | A431 | A549 | DU145 | C6 |
| 2 | 0.110 | 0.046 | 0.044 | 0.066 | 0.045 | 0.170 |
| | MDA MB435 | HT29 | PC3 | T47D | U118MG | U373MG |
| 2 | 0.054 | 0.061 | 0.049 | 0.022 | 0.059 | 0.057 |

Inhibition of the Polymerization of Tubulin

Substance 2 was tested in an in vitro test for inhibition of the polymerization of bovine tubulin. Tubulin, which had been purified by cycling polymerization and depolymerization, and which had been caused to polymerize by the addition of GTP and heating, was used in this test. The EC₅₀ values of the polymerization inhibition of tubulin with 30% associated proteins (MAPs) and of tubulin freed from MAP are given in Table 4. The results show a very potent inhibiting activity of substance 2 on the polymerization of tubulin.

TABLE 4
Inhibition of Tubulin Polymerization. Average
value of two independent experiments.

Inhibition of the Tubulin
Polymerization,
EC50 in μg/mL

| Example | with 30% MAPs | without MAPs |
|---|---|---|
| 2 | 0.85 | 2.52 |

Description of the Methods Used

XTT-Test of Cellular Dehydrogenase Activity

The adherent, growing tumor cell lines KB/HeLa, SKOV-3, SF-268, NCI-H460, ASPC1, A172, A431, A549, DU145, C6, MDAMB435, HT29, PC3, T47D, U118MG and U373MG were cultured under standard conditions in the gassing incubator at 37° C., 5% CO₂ and 95% relative humidity. On day 1 of the experiment, cells were detached with trypsin/EDTA and pelleted by centrifugation. Subsequently, the cell pellet was re-suspended in the appropriate cell count in the respective culture medium and transferred to a 96-well microtiter plate. The plates were then cultured overnight in the gassing incubator. The test substances were prepared as 1 mg/mL stock solutions in DMSO and diluted on day 2 of the experiment with culture medium to the appropriate concentrations. The substances in the culture medium were then added to the cells and incubated for 45 hours in the gassing incubator. Cells, which had not been treated with the test substance, acted as controls. For the XTT assay, 1 mg/mL XTT (sodium 3′-(1-(phenylaminocarbonyl)-3,4-tetrazolium}-bis(4-methoxy-6-nitro) benzenesulfonic acid) was dissolved in RPMI-1640 medium without phenol red. In addition, a 0.383 mg/mL PMS (N-methyl dibenzopyrazine methyl sulfate) solution in a phosphate-buffered salt solution (PBS) is prepared. On the fourth day of the experiment, 75 μL/well of XTT-PMS mixture was pipetted onto the cell plates, which had meanwhile been incubated for 45 hours with the test substances. For this purpose, the XTT solution was mixed with the PMS solution in a ratio of 50:1 (v/v) shortly before use. Subsequently, the cell plates were incubated for a further 3 hours in a gassing incubator and the optical density (OD_{490 nm}) was determined in the photometer. The percentage inhibition, relative to the control, is determined by means of the OD_{490 nm} and plotted semi-logarithmically in the form of a concentration vs. activity curve. The EC₅₀ is calculated from the concentration-activity curve by means of a regression analysis, using the Graphpad Prism program

Cell Cycle Analysis by Means of the RKOp27 Model

The assay is carried out in 96-well plates. By the inducible expression of p27^{kip1}, the growth of the cells is arrested completely, but the cells do not die. By comparing the effectiveness on cells, which have and have not been induced, conclusions can be drawn with regard to the mechanism of the action (cell cycle specificity) of the therapeutic agents. Cells, not induced, are seeded in an approximately 3 times higher cell count, since there is no longer any cell division during the assay in comparison to cells not induced (20,000 cells/well induced, 6250 cells/well not induced). The controls are untreated cells (±induction). The induction is carried out with 3 μM of Muristeron A. On the first day, the cells are exposed (±Muristeron A) and incubated for 24 hours at 37° C. On day 2, the test substance is added (control DMSO) and incubated for a further 45 hours at 37° C., before the standard DST assay is carried out.

Tubulin Polymerization Assay

The assay is carried out on the basis of a method of Bollag et al. Lyophilized bovine tubulin (Cytoskeleton, ML 113 Tubulin 30% MAPs, TL238 free of Tubulin MAP) is dissolved at a concentration of 2 mg/mL (ML1113 in 80 mM PIPES, 0.5 mM EGTA, 2 mM MgCl₂, pH 6.9, 1 mM GTP) or 5 mg/mL (TL238 in 80 mM PIPES, 1 mM EGTA, 0.5 mM MgCl₂, 20% (v/v) glycerol pH 6.9, 1 mM GTP). The test substances are diluted in 10% DMSO (v/v) and 5 μL of the dilutions is transferred to a 96-well microtiter plate (Nunc. half area plate). After the addition of 45 μL of the tubulin solution, the polymerization is determined at 340 mM in a Spectramax 190 Microtiter Plate Reader (Molecular Devices) by means of a kinetic program at 30-second intervals over a period of 20 minutes. The resulting area under the curve values are used to calculate the inhibition in relation to the untreated control and plotted semilogarithmically in the form of a concentration-activity curve. The EC₅₀ is calculated induced) by regression analysis from the concentration-activity curve using the Graphpad Prism program. The controls are untreated cells (±induction). The induction is carried out with 3 μM Muristeron A. On day 1, the cells are exposed (±Muristeron A) and incubated for 24 hours at 37° C. On day 2, the test substance (control DMSO) is added and incubated for a further 45 hours at 37° C., before a standard XTT assay is carried out.

Examples of Pharmaceutical Forms of Administration

### EXAMPLE I

Tablet with 50 mg active ingredient

| Composition: | |
|---|---|
| (1) active ingredient | 50.0 mg |
| (2) lactose | 98.0 mg |
| (3) corn starch | 50.0 mg |
| (4) polyvinylpyrrolidone | 15.0 mg |
| (5) magnesium stearate | 2.0 mg |
| Total: | 215.0 mg |

Preparation:

(1), (2) and (3) are mixed and granulated with an aqueous solution of (4). Tablets are pressed from this mixture.

### EXAMPLE II

Capsule with 50 mg active ingredient

| Composition: | |
|---|---|
| (1) active ingredient | 50.0 mg |
| (2) dried corn starch | 58.0 mg |
| (3) powdered lactose | 50.0 mg |
| (4) magnesium stearate | 2.0 mg |
| Total: | 160.0 mg |

Preparation:

(1) is titurated with (3). The product is added to the mixture of (2) and (4) with intensive mixing. The powder mixture is filled on a capsule-filling machine into hard-shell gelatin capsules.

It should be apparent to those skilled in the art that the foregoing description and examples are illustrative only and not limiting, having been presented by way of example only. All the features disclosed in this description may be replaced by alternative features serving the same purpose, and equivalents thereof. Therefore, numerous other embodiments of the modifications thereof are contemplated as falling within the scope of the present invention as defined herein and equivalents thereto.

What is claimed is:

1. A compound having the Formula 1 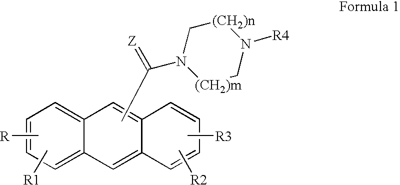
in which
R, R₁, R₂, R₃ are each independently linked to any one of the C₁ to C₁₀ aromatic carbon atoms of anthracene, wherein only one of the R, R₁, R₂, R₃ is linked to any one of the C: to C₁₀ aromatic carbon atoms, wherein R. R., R₂, R₃ alternatively are the same or different, and independently of one another, represent hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(Ca-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably allyl, (C₂-C₆)-alkinyl, preferably ethinyl or propargyl, aryl, the aryl group being unsubstituted or a linear or branched (C₁-C₈)-alkyl, substituted one or more times, similarly or differently, with halogen, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, cyano, isocyano, linear or branched (C₁-C₈)-alkoxycarbonyl, linear or branched (C₁-C₄)-alkyl substituted with one or more halogen atoms, preferably trifluoromethyl, (C₂-C₆)-alkenyl, preferably allyl, (C₂-C₆)-alkinyl, preferably ethinyl or propargyl;
Z is oxygen or sulfur, the 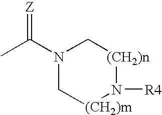
group, which is a substituent on the anthracene compound, is linked to any one of the C₁-C₁₀ carbon atoms of the ring structure;
n, m independently of one another, is a whole number from 1 to 4;
R₄ is a linear or branched (C₁-C₁₂)-alkyl group, which is saturated or unsaturated with one to three double bonds and/or triple bonds and is unsubstituted or alternatively substituted at the same or at different carbon atoms with one, two or more aryl, heteroaryl, halogen, cyano, (C₁-C₆)-alkoxycarbonylamino, (C₁-C₆)-alkoxy, amino, mono-(C₁-C₄)-alkylamino or di-(C₁-C₄)-alkylamino, a (C₆-C₁₄)-aryl group, (C₆-C₁₄)-aryl-(C₁-C₄)-alkyl group or a (C₂-C₁₀)-heteroaryl or (C₂-C₁₀)-heteroaryl-(C₁-C₄)-alkyl group, containing one or more hetero atoms selected from the group comprising nitrogen, oxygen and sulfur, the (C₁-C₄)-alkyl group being unsubstituted or substituted one or more times, identically or differently with (C₁-C₆)-alkyl or halogen and the (C₆-C₁₄)-aryl or (C₂-C₁₀)-heteroaryl group represent unsubstituted or substituted, one or more times with the same or different linear or branched (C₁-C₈)-alkyl, (C₃-C₇)-cycloalkyl, linear or branched (C₁-C₈)-alkylcarbonyl, preferably acetyl, hydroxy, linear or branched (C₁C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl.

2. A compound having Formula 1 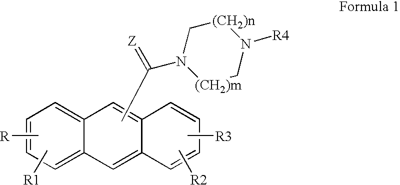
in which
R, R₁, R₂, R₃ are each independently linked to any one of the C₁ to C₁₀ aromatic carbon atoms of anthracene, wherein only one of the R, R₁, R₂, R₃ is linked to any one of the C: to C₁₀ aromatic carbon atoms, wherein R, R₁, R₂, R₃ alternatively are the same or different, and independently of one another, represent hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably allyl, (C₂-C₆)-alkinyl, preferably ethinyl or propargyl, aryl, the aryl group being unsubstituted or a linear or branched (C₁-C₈)-alkyl, substituted one or more times, similarly or differently, with halogen, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, cyano, isocyano, linear or branched (C₁-C₈)-alkoxycarbonyl, linear or branched (C₁-C₄)-alkyl substituted with one or more halogen atoms, preferably trifluoromethyl, (C₂-C₆)-alkenyl, preferably allyl, (C₂-C₆)-alkinyl, preferably ethinyl or propargyl;
Z is oxygen or sulfur, the 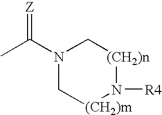
group, which is a substituent on the anthracene compound, is linked to any one of the C₁-C₁₀ carbon atoms of the ring structure;
n, m independently of one another, is a whole number from 1 to 4;
R₄ is a phenyl or a naphthyl group, which is unsubstituted or substituted once or repeatedly with the same or different linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 2-, 4-, 5- or 6-pyrimidinyl group or a 2-, 4-, 5- or 6-pyrimidinyl-(C₁-C₄)-alkyl group, the (C₁-C₄)-alkyl group being unsubstituted or substituted, once or repeatedly, identically or differently, with (C₁-C₆)-alkyl or halogen, and the 2-, 4-, 5- or 6-pyrimidinyl group being unsubstituted or substituted one to three times with the same or different, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 3-, 4-, 5- or 6-pyridazinyl group or a 3-, 4-, 5- or 6-pyridazinyl-(C₁-C₄)-alkyl group, the (C₁-C₄)-alkyl group being unsubstituted or substituted one or more times, identically or differently, with linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 2-, 3-, 5- or 6-pyrazinyl group or a 2-, 3-, 5- or 6-pyrazinyl-(C₁-C₄)-alkyl group, the (C₁-C₄)-alkyl group being unsubstituted or substituted one or more times, identically or differently, with (C₁-C₆)-alkyl, halogen and the 2-, 3-, 5-, or 6-pyrazinyl group being unsubstituted or substituted one to three times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 3-, 4-, 5-, 6-, 7- or 8-cinnolinyl group or a 3-, 4-, 5-, 6-, 7- or 8-cinnolinyl-(C₁-C₄)-alkyl group, the (C₁-C₄)-alkyl group being unsubstituted or substituted one or more times, identically or differently, with (C₁-C₆)-alkyl, halogen and the 3-, 4-, 5-, 6-, 7- or 8-cinnolinyl group being unsubstituted or substituted one to five times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 2-, 4-, 5-, 6-, 7- or 8-quinazolinyl group or a 2-, 4-, 5-, 6-, 7- or 8-quinazolinyl-(C₁-C₄)-alkyl group, the (C₁-C₄)-alkyl group being substituted one or more times, identically or differently, with hydrogen, (C₁-C₆)-alkyl, halogen, and the 2-, 4-, 5-, 6-, 7- or 8-quinazolinyl group being substituted one to five times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 2-, 3-, 5-, 6-, 7- or 8-quinoxalinyl group or a 2-, 3-, 5-, 6-, 7- or 8-quinoxalinyl-(C₁-C₄)-alkyl group, the (C₁-C₄)-alkyl group being unsubstituted or substituted one or more times, identically or differently, with (C₁-C₆)-alkyl or halogen and the 2-, 3-, 5-, 6-, 7- or 8-quinoxalinyl group being unsubstituted one to five times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 1-, 4-, 5-, 6-, 7-, or 8-phthalazinyl group or a 1-, 4-, 5-, 6-, 7-, or 8-phthalazinyl-(C₁-C₄)-alkyl group, the (C₁-C₄)-alkyl group being unsubstituted or substituted one or more times, identically or differently, with (C₁-C₆)-alkyl or halogen and the 1-, 4-, 5-, 6-, 7- or 8-phthalazinyl group being substituted one to five times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, being substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 2-, 3-, 4-, 5-, 6-, 7- or 8-quinolyl group or a 2-, 3-, 4-, 5-, 6-, 7- or 8-quinolyl-(C₁-C₄)-alkyl group, the (C₁-C₄)-alkyl group being unsubstituted or substituted one or more times, identically or differently, with (C₁-C₆)-alkyl or halogen, and the 2-, 3-, 4-, 5-, 6-, 7- or 8-quinolyl group being substituted one to six times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, being substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 1-, 3-, 4-, 5-, 6-, 7- or 8-isoquinolyl or a 1-, 3-, 4-, 5-, 6-, 7- or 8-isoquinolyl-(C₁-C₄)-alky group, the (C₁-C₄)-alkyl group being unsubstituted or substituted one or more times, identically or differently, with (C₁-C₆)-alkyl or halogen, and the 1-, 3-, 4-, 5-, 6-, 7- or 8-isoquinolyl group being substituted one to six times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, being substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 2-, 6-, 8- or 9-[9H]-purinyl group or 2-, 6-, 8- or 9-[9H]-purinyl-(C₁-C₄)-alkyl group, the (C₁-C₄)-alkyl group being unsubstituted or substituted one or more times, identically or differently, with (C₁-C₆)-alkyl or halogen, and the 2-, 6-, 8- or 9-[9H]-purinyl group being substituted one to three times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 2-, 6-, 7- or 8-[7H]-purinyl group or 2-, 6-, 7- or 8-[7H]-purinyl-(C₁-C₄)-alkyl group, the (C₁-C₄)-alkyl group being unsubstituted or substituted one or more times, identically or differently with (C₁-C₆)-alkyl or halogen, and the 2-, 6-, 7- or 8-[7H]-purinyl group being substituted one to three times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 1-, 2-, 3-, 4-, 5-, 6-, 7-, 8- or 9-acridinyl or a 1-, 2-, 3-, 4-, 5-, 6-, 7-, 8- or 9-acridinyl-(C₁-C₄)-alkyl group, the (C₁-C₆)-alkyl group being unsubstituted or substituted one or more times, identically or differently, with (C₁-C₆)-alkyl or halogen and the 1-, 2-, 3-, 4-, 5-, 6-, 7-, 8- or 9-acridinyl group being substituted one to eight times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 1-, 2-, 3-, 4-, 5-, 6-, 7-, 8 or 9-phenanthridinyl or a 1-, 2-, 3-, 4-, 5-, 6-, 7-, 8- or 9-phenanthridinyl-(C₁-C₆)-alkyl group, the (C₁-C₆)-alkyl group being unsubstituted or substituted one or more times, identically or differently with hydrogen, (C₁-C₆)-alkyl, or halogen, and the 1-, 2-, 3-, 4-, 5-, 6-, 7-, 8- or 9-phenanthridinyl group being unsubstituted or substituted one to eight times, identically or differently with linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 2-, 3-, 4-, 5- or 6-pyridyl group, the 2-, 3-, 4-, 5- or 6-pyridinyl group being substituted one to four times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 2-, 3-, 4-, 5- and 6-pyridyl-(C₁-C₆)-alkyl group, the (C₁-C₆)-alkyl group being unsubstituted or substituted one or more times, identically or differently, with (C₁-C₆)-alkyl or halogen, and the 2-, 3-, 4-, 5- or 6-pyridinyl group being substituted one to four times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 2-, 3-, 4- or 5-thienyl group or a 2-, 3-, 4- or 5-thienyl-(C₁-C₆)-alkyl group, the (C₁-C₆)-alkyl group being unsubstituted or substituted one or more times, identically or differently, with (C₁-C₆)-alkyl or halogen and the 2-, 3-, 4- or 5-thienyl group being substituted one to three times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 2-, 4- or 5-thiazolyl group or a 2-, 4- or 5-thiazolyl-(C₁-C₆)-alkyl group, the (C₁-C₆)-alkyl group being unsubstituted or substituted one or more times, identically or differently, with (C₁-C₆)-alkyl or halogen and the 2-, 4- or 5-thiazolyl group being substituted once or twice, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, with or more substituted halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 3-, 4- or 5-isothiazolyl group or a 3-, 4- or 5-isothiazolyl-(C₁-C₆)-alkyl group, the (C₁-C₆)-alkyl group being unsubstituted or substituted one or more times, identically or differently with (C₁-C₆)-alkyl or halogen, and the 3-, 4- or 5-isothiazolyl group being substituted once or twice, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 2-, 4-, 5-, 6- or 7-benzthiazolyl group or a 2-, 4-, 5-, 6- or 7-benzthiazolyl-(C₁-C₆)-alkyl group, the (C₁-C₆)-alkyl group being unsubstituted or substituted one or more times, identically or differently, with (C₁-C₆)-alkyl or halogen, and the 2-, 4-, 5-, 6- or 7-benzthiazolyl group being substituted one to four times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 1-, 2-, 4- or 5-imidazolyl group or a 1-, 2-, 4- or 5-imidazolyl-(C₁-C₆)-alkyl group, the (C₁-C₆)-alkyl group being unsubstituted or substituted one or more times, identically or differently, with (C₁-C₆)-alkyl or halogen and the 1-, 2-, 4- or 5-imidazolyl group being substituted one to three times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 1-, 3-, 4- or 5-pyrazolyl group or a 1-, 3-, 4- or 5-pyrazolyl-(C₁-C₆)-alkyl group, the (C₁-C₆)-alkyl group being unsubstituted or substituted one or more times, identically or differently, with (C₁-C₆)-alkyl or halogen, and the 1-, 3-, 4- or 5-pyrazolyl group being substituted one to three times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 1-, 2-, 3-, 4- or 5-pyrrolyl group or a 1-, 2-, 3-, 4- or 5-pyrrolyl-(C₁-C₆)-alkyl group, the (C₁-C₆)-alkyl group being unsubstituted or one or more times, identically or differently, with (C₁-C₆)-alkyl or halogen, and the 1-, 2-, 3-, 4- or 5-pyrrolyl group being substituted one to four times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, with or more substituted halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 1-, 3- or 5-[1,2,4]-triazolyl group or 1-, 3- or 5-[1,2,4]-triazolyl-(C₁-C₆)-alkyl group, the (C₁-C₆)-alkyl group being substituted one or more times, identically or differently, with hydrogen, (C₁-C₆)-alkyl or halogen and the 1-, 3- or 5-[1,2,4]-triazolyl group, unsubstituted or substituted once or twice, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 1,4- or 5-[1.2.3]-triazolyl group or a 1-, 4- or 5-[1.2.3]-triazolyl-(C₁-C₆)-alkyl group, the (C₁-C₆)-alkyl group being unsubstituted or substituted one or more times, identically or differently, with (C₁-C₆)-alkyl or halogen and the 1-, 4- or 5-[1.2.3]-triazolyl group being substituted once or twice, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 1- or 5-[1H]-tetrazolyl group or a 1- or 5-[1H]-tetrazolyl-(C₁-C₆)-alkyl group, the (C₁-C₆)-alkyl group being unsubstituted or substituted one or more times, identically or differently, with (C₁-C₆)-alkyl or halogen, and the 1- or 5-[1H]-tetrazolyl group being substituted with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 2- or 5-[2H]-tetrazolyl group or a 2- or 5-[2H]-tetrazolyl-(C₁-C₆)-alkyl group, the (C₁-C₆)-alkyl group being unsubstituted or substituted one or more times, identically or differently, with (C₁-C₆)-alkyl or halogen, and the 2- or 5-[2H]-tetrazolyl group being substituted with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 2-, 4- or 6-[1.3.5]-triazinyl group or a 2-, 4- or 6-[1.3.5]-triazinyl-(C₁-C₆)-alkyl group, the (C₁-C₆)-alkyl group being unsubstituted or substituted one or more times, identically or differently, with hydrogen, (C₁-C₆)-alkyl or halogen and the 2-, 4- or 6-[1.3.5]-triazinyl group being substituted once or twice, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, with or more substituted halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 2-, 4- or 5-oxazolyl group or a 2-, 4- or 5-oxazolyl-(C₁-C₆)-alkyl group, the (C₁-C₆)-alkyl group being unsubstituted or substituted one or more times, identically or differently, with (C₁-C₆)-alkyl or halogen, and the 2-, 4- or 5-oxazolyl group being substituted once or twice, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 3-, 4- or 5-isoxazolyl group or a 3-, 4- or 5-isoxazolyl-(C₁-C₆) group, the (C₁-C₆)-alkyl group being unsubstituted or substituted one or more times, identically or differently, with (C₁-C₆)-alkyl or halogen, and the 3-, 4- or 5-isoxazolyl group being substituted once or twice, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl;
a 1-, 2-, 3-, 4-, 5-, 6- or 7-indolyl group or a 1-, 2-, 3-, 4-, 5-, 6- or 7-indolyl-(C₁-C₆)-alkyl group, the (C₁-C₆)-alkyl group being unsubstituted or substituted one or more times, identically or differently, with (C₁-C₆)-alkyl or halogen, and the 1-, 2-, 3-, 4-, 5-, 6- or 7-indolyl group being substituted one to six times, identically or differently, with hydrogen, linear or branched (C₁-C₈)-alkyl, (C₃-C₇)-cycloalkyl, linear or branched (C₁-C₈)-alkylcarbonyl, preferably acetyl, hydroxy, linear or branched (C₁-C₈)-alkoxy, preferably methoxy and ethoxy, halogen, linear or branched aryl-(C₁-C₈)-alkoxy, preferably benzyloxy or phenylethyloxy, trityloxy, trimethylsilyloxy, amino, mono-(C₁-C₄)-alkylamino, di-(C₁-C₄)-alkylamino, (C₂-C₅)-cycloalkylamino, morpholino, heterocyclic-(C₁-C₆)-alkoxy, carboxy, imidocarboxy, carboxamidine, linear or branched (C₁-C₈)-alkoxycarbonylamino, linear or branched (C₁-C₈)-alkylcarbonylamino, preferably acetamino, sulfonyloxy, sulfenyloxy, sulfinyloxo, nitro, nitroso, thio, linear or branched (C₁-C₈)-alkylthio, linear or branched (C₁-C₈)-alkylsulfo, linear or branched (C₁-C₈)-alkylsulfoxy, cyano, isocyano, linear or branched cyano-(C₁-C₆)-alkyl, preferably cyanomethyl, linear or branched (C₁-C₈)-alkoxycarbonyl, linear or branched (C₁-C₄)-alkyl, substituted with one or more halogen atoms, 1 preferably trifluoromethyl, linear or branched carboxy-(C₁-C₈)-alkyl, linear or branched (C₁-C₈)-alkoxycarbonyl-(C₁-C₆)-alkyl, (C₂-C₆)-alkenyl, preferably, allyl, (C₂-C₆)-alkinyl, preferably ethinyl or propargyl.

3. The compound of claim 2, wherein R₄ is phenyl which is unsubstituted or substituted one to five times with identical or different C₁-C₆ alkoxy groups.

4. The compound of claim 3, wherein oxygen atoms contained in substituents on adjacent aromatic ring carbon atoms are linked by a C₁-C₂ alkylene group.

5. The compound of claim 2, wherein R₄ is 3,4-dimethoxyphenyl.

6. The compound of claim 2, wherein R, R₁, R₂ and R₃ are each hydrogen, m is 1 and n is 1 or 2.

7. The compound of claim 2, wherein R, R₁, R₂, R₃ are each hydrogen, m and n are 1 and R₄ is 3,5-dimethoxyphenyl.

8. The compound as in claim 1, wherein the compound contains at least one asymmetric center, and is in the form of racemates, or is in the form of pure enantiomers or diastereoisomers, or is in the form of mixtures of enantiomers, or is in the form of mixtures of diastereoisomers, or is in the form of mixtures of enantiomers and diastereoisomers, or is in the form of tautomers of any of the foregoing.

9. The compound as in claim 2, wherein the compound contains at least one asymmetric center, and is in the form of racemates, or is in the form of pure enantiomers or diastereoisomers, or is in the form of mixtures of enantiomers, or is in the form of mixtures of diastereoisomers, or is in the form of mixtures of enantiomers and diastereoisomers, or is in the form of tautomers of any of the foregoing.

10. A compound having one of the following formulas:
Anthracene-9-yl-[4-(4-nitro-phenyl)-piperazine-1-yl]-methanone;
Anthracene-9-yl-[4-(3,5-dimethoxy-phenyl)-piperazine-1-yl]-methanone;
Anthracene-9-yl-[4-phenyl)-piperazine-1-yl]-methanone;
Anthracene-9-yl-(4-naphthalene-1-yl-piperazine-1-yl)-methanone;
Anthracene-9-yl-(4-biphenyl-2-yl-piperazine-1-yl)-methanone;
Anthracene-9-yl-[4-(3-hydroxy-phenyl)-piperazine-1-yl]-methanone;
Anthracene-9-yl-[4-(4-trifluoromethyl-pyridine-2-yl)-piperazine-1-yl]-methanone;
Anthracene-9-yl-[4-(6-methyl-pyridine-2-yl)-piperazine-1-yl]-methanone;
Anthracene-9-yl-(2,3,5,6-tetrahydro-[1,2′]-bipyrazinyl-4-yl)-methanone; 2-[4-(Anthracene-9-carbonyl)-piperazine-1-yl]-nicotinnitrile;
Anthracene-9-yl-[4-(5-trifluoromethyl-pyridine-2-yl)-piperazine-1-yl]-methanone; and
Anthracene-9-yl-(4-pyridine-2-yl-piperazine-1-yl)-methanone.

11. A method for making an anthracene compound comprising contacting an acridinecarboxylic acid of Formula 2, wherein Z is oxygen or sulfur, and Y is a leaving group selected from the group of halogen, C₁-C₆ alkoxy, —O-tosyl, —O-mesyl, tetrazolyl and imidazolyl, 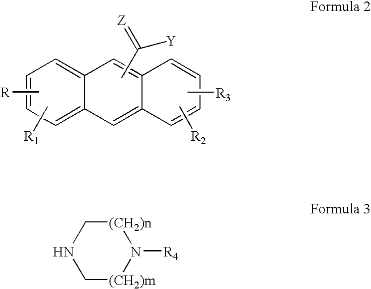
with an amine of Formula 3.

12. The method of claim 11 further comprising a condensing agent, a catalyst, a diluent, and an adjuvant.

13. A pharmaceutical composition comprising a pharmaceutically effective amount of a compound as in claim 1 for treatment of a tumor in a mammal.

14. The composition of claim 13, further comprising an adjuvant, an additive, and a carrier.

15. A pharmaceutical composition comprising a pharmaceutically effective amount of a compound as in claim 2 for treatment of a tumor in a mammal.

16. The composition of claim 15, further comprising an adjuvant, an additive, and a carrier.

17. A method for treatment of a benign or malignant tumor in a mammal comprising administering a pharmaceutically effective dose of a compound of claim 1 to a mammal.

18. A method for treatment of a benign or malignant tumor in a mammal comprising administering a pharmaceutically effective dose of a compound of claim 2 to a mammal.
